SEQUENCE LISTING (11 sequences)

SEQ ID NO: 1 - DNA, 438 bp, Glycine max
atgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcattttag

SEQ ID NO: 2 - DNA, 1362 bp, Glycine max
atggaaaggagcagcgtcatggttattggactgagtgtgcatagtacgcctgtggaaatgcgtgaaaagcttgccataccagaagcagaatggcctcgagccattgcggagctttgtagtctgaatcacattgaggaagcagctgttctgagcacctgcaacagaatggaaatatatgttgttgctctgtccaagcaccgtggtgttaaagaagtcactgaatggatgtccaaaacaagtgggattccagttgcagatctttgccagcatcagtttctgctatacaacaaagatgccacacaacacctttttgaagtatctgcaggtcttgattctctagtgttgggagaaggtcaaatccttgcccaggtgaagcaggttgtcaaagttggacaaggagtgaatggctttgggaggaacattagtggactattcaagcatgcaatcactgtggggaagagggttagaactgagacaaacattgctgctggtgcagtttctgttagctcagcagctgttgaactggccctgatgaagctacctgaagcctcacatgccaatgcaaggatgttggtcattggagctgggaagatgggaaagcttgtgatcaagcatttggtggcaaaagggtgcacaaagatggtggttgtcaatagaagtgaggagagagttgccgcgatccgtgaagaaatcaaggatgttgagataatctacaagccactctcggagatgctcacatgcattggtgaggcagatgtggttttcaccagcacagcatcagagaatccactgttcttgaaggatgatgtcaaggaacttcctcccgccaccgatgaagtcggcggccatcgccttttcgtcgatatatctgttcctaggaatgttggatcatgtctctcagaccttgagtctgtgagagtgtacaatgttgatgaccttaaggaggttgtggcagccaacaaagaggataggctaagaaaagccatggaggctcaagcaatcattggtgaagaatcaaaacaatttgaggcttggagagactcattggaaactgttcctaccattaaaaagttgagggcatatgctgaaagaataaggcttgctgagcttgagaagtgcctaggtaagatgggtgatgatatcaacaagaagacacaaagagctgtggatgatcttagcaggggtatagtgaataagttgcttcatgggccaatgcaacacttgaggtgtgatggcagtgacagcaggactctaagtgagacccttgagaacatgcatgctttgaatagaatgttcaaccttgagactgaaatatctgttttggagcagaagattcgagccaaggtggagcaaaagccatag

SEQ ID NO: 3 - DNA, 177 bp, Artificial
  synthetic oligonucleotide
atggcttcctcagttctttcctctgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcag

SEQ ID NO: 4 - DNA, 186 bp, Artificial
  synthetic oligonucleotide
atgatgagagcgcggttcccattactgttgctgggagttgttttcctagcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaag

SEQ ID NO: 5 - DNA, 1224 bp, Glycine max
atggttcaagatttttctgatacacctcttattggtgatgataagattggagtgttattgttgaacctcggaggtccagagactttagaagatgtgcagccttttttgtttaaccttttcgctgacccagatattatacgacttccaaggttatttagctttcttcaaaagccattggcccaatttgtatctgttttaagagcaccaaagagcaaagaaggctatgcttcaattggtggtggatctcctcttagacgtataactgatgcacaggctgaagagttgaggaaatctctttggtcaaagaatgtcccagccaaagtgtatgttggcatgcgttactggcatccattcaccgaagaggctattgaacagattaaaagggatggaattacaaagcttgttgtgcttccactttatccacaattttcaatttcaaccagtggttcaagtcttcgtctcttggagagtatattcagagatgatgagtatctagtcaacatgcagcacacagtaattccttcatggtaccagcgtgaaggatacattaaggccatgacaaatttaattgaaaaggagctaagaggttttgactgccctgaggaggtcatgatattctttagtgcacatggggtgcctcttgcttatgtagaagaggctggtgatccatacaaggcagagatggaggaatgtgttgatttgatcatggaagaacttgaaaagagaaagataactaatgcatacacccttgcttatcagagtagagttggacctgtggaatggttaaaaccttatacagatgagacaataattgaacttggggaaaagggagtaaaaagtctgctggctgtaccaattagctttgtcagcgagcatattgaaactctagaagaaattgatgttgagtacaaagaattggctctaaactctggtatagaaaagtggggtcgtgttcctgctctaggaactgaacccactttcatttcagatttagcagatgctgtaattgagagtctcccttatgttggtgcaatggcggtttcaaaccttgaagctcgacagtctttggttccactgggcagtgtagaagagttattggcagcatatgactcgcaacgtagggagttgccgccaccagtaattgtgtgggaatggggatggaccaaaagtgctgaaacttggaatggaagacaccagtggggaatattgcccttgtttaggtga

SEQ ID NO: 6 - DNA, 1287 bp, Glycine max
atggttcaagatttttctgatacacctcttattggtgatgataagattggagtgttattgttgaacctcggaggtccagagactttagaagatgtgcagccttttttgtttaaccttttcgctgacccagatattatacgacttccaaggttatttagctttcttcaaaagccattggcccaatttgtatctgttttaagagcaccaaagagcaaagaaggctatgcttcaattggtggtggatctcctcttagacgtataactgatgcacaggctgaagagttgaggaaatctctttggtcaaagaatgtcccagccaaagtgtatgttggcatgcgttactggcatccattcaccgaagaggctattgaacagattaaaagggatggaattacaaagcttgttgtgcttccactttatccacaattttcaatttcaaccagtggttcaagtcttcgtctcttggagagtatattcagagatgatgagtatctagtcaacatgcagcacacagtaattccttcatggtaccagcgtgaaggatacattaaggccatgacaaatttaattgaaaaggagctaagaggttttgactgccctgaggaggtcatgatattctttagtgcacatggggtgcctcttgcttatgtagaagaggctggtgatccatacaaggcagagatggaggaatgtgttgatttgatcatggaagaacttgaaaagagaaagataactaatgcatacacccttgcttatcagagtagagttggacctgtggaatggttaaaaccttatacagatgagacaataattgaacttggggaaaagggagtaaaaagtctgctggctgtaccaattagctttgtcagcgagcatattgaaactctagaagaaattgatgttgagtacaaagaattggctctaaactctggtatagaaaagtggggtcgtgttcctgctctaggaactgaacccactttcatttcagatttagcagatgctgtaattgagagtctcccttatgttggtgcaatggcggtttcaaaccttgaagctcgacagtctttggttccactgggcagtgtagaagagttattggcagcatatgactcgcaacgtagggagttgccgccaccagtaattgtgtgggaatggggatggaccaaaagtgctgaaacttggaatggaagagcagctatgcttgctgtgcttcttctgttgtttttagaagtcaccactggtgaagggtttctgcaccagtggggaatattgcccttgtttaggtga

SEQ ID NO: 7 - DNA, 918 bp, Glycine max
atgcttcttcaaacgaggcaatttggcacagcgtgtccatgggtcagactcacacccgcaagcaaaagaaatagctcagtctcattgagaaggagcaattgtggcccaaaatgttcggtgtcccaattggaattgaagagtgagagtgagagcaaaacgaagccgtatccagcagaggcatcgaggaccataatggagttggctaaggtgggcacgctgtgtacgttgacccaagagggtttgccccttggtactggggttcgattcgcggttgaccctgaacatggcactcccttgttctgcttcaattccactgacaacactaacatcccctcttctcttcatgttcagtttgaacaatttggattgcgcactcctcagtgcactattcaaggaacccttaccaaaccacaggatccaaagcgtcttgtttctgtgtggaggaaaaggtttggagaagaagttgatcgagattttatttacattattgccgtggatcgggtactccaattggaagacattcaggaggatggcatatgggtcacctcttcagattacaaaaatgctcaacccgatcctcttcgagactctgcacacaacttagtcactgaaatcaacaccaacaatatggaagacattactcgcttttgcaatgtctacgttgatttggatttcctggtctccgaggcaaagatgctatgggttgatcgcttgggctttgacatgcgtttgtcttcccctcacaaaggcgtgtttgacgtccgcattcctttccccagagaagtcaccgatgagaaaggtgccaagtcgacgtttaattgtatgtcacaactcgcttgggaggtagaaagaaacttccaacatccagacttttcaaaggttaaagagttgaagccagtcaagtccccttttctgtaa

SEQ ID NO: 8 - DNA, 1230 bp, Bradyrhizobium japonicum
atggattacgcccagttcttcaataccgccctcgatcgtctccacaccgagcggcgctaccgcgtgttcgccgatctcgaacgcatcgccggccggttcccacatgcgctgtggcactcgcccaagggcaaacgcgacgtcgtgatctggtgctccaacgattatctcggcatgggccagcacccgaaggtggtcggcgccatggtcgaaaccgcgacccgcgtcggcaccggcgcaggcggcacccgcaacatcgccggcacgcatcatccgctggtacagctcgaggccgagctcgccgatctccacggcaaggaagcgtcgctgctgttcacctcgggctatgtctcgaaccagaccggcatcgcgaccatcgcgaagctcattccgaactgcctgatcctgtcggacgagctcaaccacaattcgatgatcgagggcatccgccagtccggctgcgagcgcgtcgtcttccgccacaacgacctcgccgacctcgaagagaagttgaaggccgcaggtccgaaccggccgaagctgatcgcctgcgagagcctctattcgatggacggcgacgtcgccccgctcgccaagatctgcgatctcgccgagaaatatggcgcgatgacctatgtcgacgaggtccacgccgtcggcatgtacggcccgcgcggcggcggcatcgccgagcgtgacggcgtcatgcatcgcatcgacattctcgaaggcacgctggccaaggcgttcggctgcctcggcggctacatcgccgccaacggccagatcatcgacgccgtgcgctcctacgcgccgggcttcatcttcaccaccgcgctgccgccggcgatctgctcggccgcgaccgccgcgatcaggcatctgaagacctcgaactgggagcgcgagcgccatcaggaccgcgccgcccgcgtcaaggcgatcctcaacgccgccggcctgcccgtgatgtcgagcgacacccatatcgtaccgctgttcatcggcgatgccgagaagtgcaagcaggcgtcggacctcctgctggaacagcacggcatctacattcagccgatcaactatccgacggtagccaagggcaccgaacgcctgcgcatcacgccctcgccctatcacgacgacggcttgatcgatcagctcgccgaagcgctcctgcaggtgtgggaccgcctgggtctgccgctcaagcaaaagtcgctggcggcggagtag

SEQ ID NO: 9 - protein, 531 aa, Glycine max
MNSPIHAPSPPSSSSSCSYNRPPPCLAHASRNFKFPLLLPQAIRTSQKMYRCSGGHVEGSTNTNPLKNYVVGRSTSGWFETQPLVSEQLLNRRLLPVEALVTPTVQDFSDTPLIGDDKIGVLLLNLGGPETLEDVQPFLFNLFADPDIIRLPRLFSFLQKPLAQFVSVLRAPKSKEGYASIGGGSPLRRITDAQAEELRKSLWSKNVPAKVYVGMRYWHPFTEEAIEQIKRDGITKLVVLPLYPQFSISTSGSSLRLLESIFRDDEYLVNMQHTVIPSWYQREGYIKAMTNLIEKELRGFDCPEEVMIFFSAHGVPLAYVEEAGDPYKAEMEECVDLIMEELEKRKITNAYTLAYQSRVGPVEWLKPYTDETIIELGEKGVKSLLAVPISFVSEHIETLEEIDVEYKELALNSGIEKWGRVPALGTEPTFISDLADAVIESLPYVGAMAVSNLEARQSLVPLGSVEELLAAYDSQRRELPPPVIVWEWGWTKSAETWNGRAAMLAVLLLLFLEVTTGEGFLHQWGILPLFR

SEQ ID NO: 10 - protein, 4 aa, Artificial
  synthetic peptide
NPIR

SEQ ID NO: 11 - protein, 21 aa, Artificial
  synthetic peptide
AAMLAVLLLLFLEVTTGEGFL

(19) United States
(12) Patent Application Publication
(10) Pub. No.: US 2019/0292217 A1
(43) Pub. Date: Sep. 26, 2019

(54) TRANSGENIC PLANTS WITH UPREGULATED HEME BIOSYNTHESIS

(71) Applicant: Impossible Foods Inc., Redwood City, CA (US)

(72) Inventors: Simon Christopher Davis, San Francisco, CA (US); Michael Lassner, Portland, OR (US)

(21) Appl. No.: 16/465,999

(22) PCT Filed: Dec. 1, 2017
(86) PCT No.: PCT/US2017/064274

§ 371 Date: May 31, 2019

Related U.S. Application Data
(60) Provisional application No. 62/429,565, filed on Dec. 2, 2016.

(51) Int. Cl. C07H 21/04; C12N 15/82; C12N 15/52

(52) U.S. Cl. CPC C07H 21/04; C12N 15/52; C12N 15/8237; C12N 15/8234

(57) ABSTRACT

This disclosure generally relates to transgenic plants that recombinantly express nucleic acid sequences encoding polypeptides capable of upregulating heme biosynthesis, and methods of producing a heme-loaded heme polypeptide.

### CROSS-REFERENCE TO RELATED APPLICATIONS

This application claims the benefit of U.S. Application Ser. No. 62/429,565, filed Dec. 2, 2016. The disclosure of the prior application is considered part of and is incorporated by reference in the disclosure of this application.

### TECHNICAL FIELD

This disclosure relates to methods and materials for upregulating heme biosynthesis in plants, and more particularly, to methods for recombinantly producing heme-loaded heme polypeptides in transgenic plants, plant cells, or seeds having upregulated heme biosynthesis.

### BACKGROUND

Plants make large amounts of tetrapyrrole molecules including chlorophyll, sirohemes and heme B. The tetrapyrrole biosynthetic pathway provides key co-factors and pigments for processes such as growth and essential redox reactions. Plants control tetrapyrrole synthesis by a number of routes, including a diurnal switch, a redox response system and negative feedback loops to prevent the formation of reactive oxidative species such as photoreactive intermediates, and to accurately distribute metabolic intermediates amongst end products of the pathway. (Mochizuki et al., Trends Plant Sci 15 (9): 488-498, 2010).

### SUMMARY

This document is based on methods and materials for making transgenic plants, transgenic plant cells, and transgenic seeds in which heme biosynthesis is specifically upregulated in order to increase the heme loading of recombinant heme polypeptides produced in the transgenic plants, cells, and seeds. One molecule of the heme co-factor is typically synthesized for each polypeptide that is made. Therefore, in order to increase the specific production of heme B for incorporation into heme-containing proteins, it is important to separate the production of heme from the control mechanisms of the tetrapyrrole biosynthesis pathway, and specifically upregulate heme biosynthesis and not the other tetrapyrroles. “Up-regulation” in the context of heme biosynthesis refers to increased biosynthesis of heme without a corresponding increase in the biosynthesis of other tetrapyrroles.

In one aspect, transgenic plants and plant cells are provided that include at least one recombinant nucleic acid, wherein the recombinant nucleic acid in the transgenic plant or plant cell includes (i) a first promoter operably linked to a nucleic acid encoding a heme-containing polypeptide and; (ii) a second promoter operably linked to a nucleic acid encoding a polypeptide that upregulates heme biosynthesis. Heme-loading of the heme-containing polypeptide is increased in the transgenic plant relative to that of a corresponding control plant that does not include the polypeptide that upregulates heme biosynthesis. The first and second promoters can be the same. The first and second promoters can be inducible. The transgenic plant can be a soy plant or a rice plant. The transgenic plant can be selected from the group consisting of: a rye, a beet, a sugar beet, a parsnip, a bean such as an adzuki, a mung, a pea, a peanut, a lentil, or a garbanzo, a leafy vegetable such as an alfalfa, an arugula, a mustard, or a Brassica, a tuber such as a potato, a sweet potato, or a cassava, and a grass such as a barley, a wheat, a corn, oat, triticale or spelt. The heme-loaded heme-containing polypeptide can include at least 0.01% of total seed protein (e.g., at least 1%, 5%, or 10%) in seeds of the transgenic plant.

The first and second promoters can be seed specific promoters. In some embodiments, the seed specific promoter is selected from the group consisting of a soy beta-conglycinin seed specific promoter, a G1-Glycinin seed specific promoter, a Kunitz trypsin inhibitor (KTI) promoter, and an oleosin promoter.

In some embodiments, the recombinant nucleic acid further includes a first targeting sequence operably linked to the nucleic acid encoding the heme-containing polypeptide, and a second targeting sequence operably linked to the nucleic acid encoding the polypeptide that upregulates heme biosynthesis. The first and second targeting sequences can target the polypeptides to the same intracellular location within the transgenic plant. The first and second targeting sequences can be the same.

In some embodiments, the targeting sequence encodes a vacuole targeting signal peptide. In some embodiments, the vacuole targeting signal peptide is a soy conglycinin vacuole targeting signal peptide, a soy glycinin vacuole targeting signal peptide, or a plant seed storage protein vacuole targeting signal peptide.

In some embodiments, the targeting sequence encodes a plastid targeting signal peptide. In some embodiments, the plastid targeting signal peptide is a RuBisCO signal peptide.

In some embodiments, the targeting sequence is a soy beta-conglycinin targeting sequence. In some embodiments, the targeting sequence is a G1-Glycinin targeting sequence.

In some embodiments, the polypeptide that upregulates heme biosynthesis is a ferrochelatase, a glutamyl-tRNA reductase (GluTR) binding protein, a truncated glutamate tRNA reductase protein (GTR), an aminolevulinic acid synthase, or a combination of two or more of the polypeptides. The polypeptide that upregulates heme biosynthesis can be endogenous to the transgenic plant. The polypeptide that upregulates heme biosynthesis can be heterologous to the transgenic plant. The ferrochelatase can be a barley ferrochelatase, a tobacco ferrochelatase, a soy ferrochelatase, or a microbial ferrochelatase such as a Bradyrhizobium ferrochelatase or an Aspergillus ferrochelatase. The aminolevulinic acid synthase is a bacterial aminolevulinic acid synthase.

The heme-containing polypeptide can be a globin polypeptide in Pfam 00042. For example, the globin polypeptide can be a plant globin polypeptide. For example, the globin polypeptide can be a leghemoglobin, a non-symbiotic hemoglobin, an androglobin, a cytoglobin, a globin E, a globin X, a globin Y, a hemoglobin, a myoglobin, an erythrocruorin, a beta hemoglobin, an alpha hemoglobin, a protoglobin, a cyanoglobin, a cytoglobin, a histoglobin, a neuroglobin, a chlorocruorin, a truncated hemoglobin, a truncated 2/2 globin, a hemoglobin 3, a cytochrome, or a peroxidase.

The globin polypeptide can be expressed in the cytosol of the seeds of the transgenic plant cells. The globin polypeptide can be expressed in the vacuole of the seeds of transgenic plant cells.

This document also provides transgenic plants that include any of the plant cells described herein.

This document also provides seeds from any transgenic plants described herein, wherein the seed comprises the recombinant nucleic acid.

This document also provides progeny of the transgenic plants described herein, the progeny comprising the recombinant nucleic acid construct.

Also provided herein are methods of producing a heme-loaded heme polypeptide. The method can include growing any of the plant cell described herein, wherein heme-loading of the heme-containing polypeptide is increased in the plant cell as compared to a control plant cell that does not comprise the polypeptide that upregulates heme biosynthesis.

Provided herein are methods of producing a heme-loaded heme-containing polypeptide. The method can include germinating any of the transgenic seeds described herein in a contained system to produce a transgenic seedling and isolating the heme-loaded heme-containing polypeptide from the seedling. Heme-loading of the heme-containing polypeptide can be increased in the transgenic seedling relative to that of a corresponding seedling that does not include the polypeptide that upregulates heme biosynthesis. The contained system can be a malting system or a hydroponic system.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The word “comprising” in the claims may be replaced by “consisting essentially of” or with “consisting of,” according to standard practice in patent law.

### DESCRIPTION OF DRAWINGS

FIG. 1 is a schematic of tetrapyrrole synthesis.

FIG. 2 contains the nucleic acid sequence encoding the Leghemoglobin (Lbc2) from Glycine max (SEQ ID NO:1), the nucleic acid sequence encoding a truncated glutamyl tRNA reductase from Glycine max (corresponding to amino acids 1, and 91 to 542 (end) of Uniprot Q9ZPK4_SOYBN-Glutamyl tRNA reductase) (SEQ ID NO: 2), the nucleic acid sequence encoding the chloroplast ribulose-1-5-bisphosphate carboxylase/oxygenase small unit plastid targeting sequence (SEQ ID NO: 3), a nucleic acid sequence encoding a vacuole targeting signal sequence (Conglycinin signal peptide) (SEQ ID NO: 4), a nucleic acid sequence encoding a soluble ferrochelatase from Glycine max (SEQ ID NO: 5, the nucleic acid encodes a polypeptide having residues 105 to 500 and 522 to 531 of SEQ ID NO:9), a nucleic acid sequence encoding a full-length ferrochelatase from Glycine max (SEQ ID NO: 6, encodes residues 105 to 531 of the amino acid sequence set forth in SEQ ID NO: 9), a nucleic acid sequence encoding a glutamyl tRNA reductase binding protein from Glycine max (SEQ ID NO: 7), a nucleic acid sequence encoding a 5-aminolevulinic acid synthase from Bradyrhizobium japonicum (SEQ ID NO: 8), and an amino acid sequence of a ferrochelatase from Glycine max (Uniprot I1K551, SEQ ID NO: 9).

### DETAILED DESCRIPTION

In general, this document provides methods and materials for making and using transgenic plants to increase the specific biosynthesis of heme for incorporation into heme-containing proteins. The transgenic plants, cells, and seeds described herein include at least one recombinant nucleic acid that includes a) a promoter operably linked to a nucleic acid encoding a heme-containing polypeptide and b) a promoter operably linked to a nucleic acid that encodes a polypeptide that specifically upregulates heme biosynthesis. “Polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

To specifically upregulate heme biosynthesis, one or more of the following polypeptides can be expressed in the transgenic plant: a ferrochelatase, a glutamyl-tRNA reductase (GluTR) binding protein, a truncated glutamate tRNA reductase protein (GTR), or an aminolevulinic acid synthase. For example, a transgenic plant may express one, two, three, or four of such polypeptides. For example, a transgenic plant may express a ferrochelatase, a ferrochelatase and a GluTR binding protein, a ferrochelatase and a truncated GTR, a ferrochelatase and an aminolevulinic acid synthase, a GluTR binding protein, a GluTR binding protein and a truncated GTR, a GluTR binding protein and an aminolevulinic acid synthase, a truncated GTR, a truncated GTR and an aminolevulinic acid synthase, a ferrochelatase, a GluTR binding protein and a truncated GTR, a ferrochelatase, a GluTR binding protein and an aminolevulinic acid synthase, a GluTR binding protein, a truncated GTR, and an aminolevulinic acid synthase, or a ferrochelatase, a GluTR binding protein, a truncated GTR, and an aminolevulinic acid synthase.

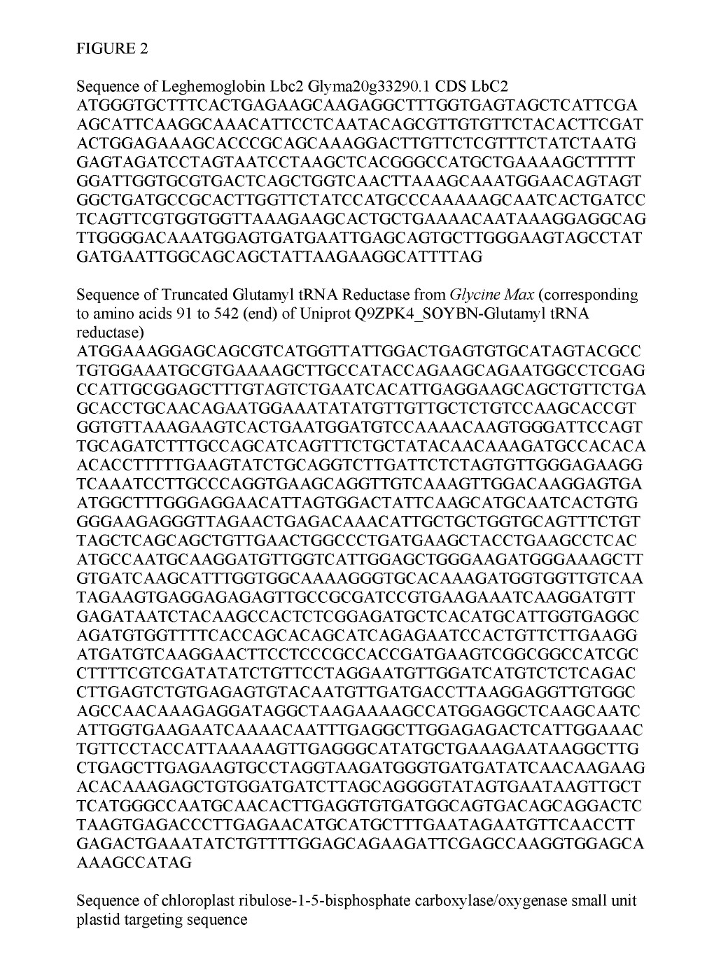

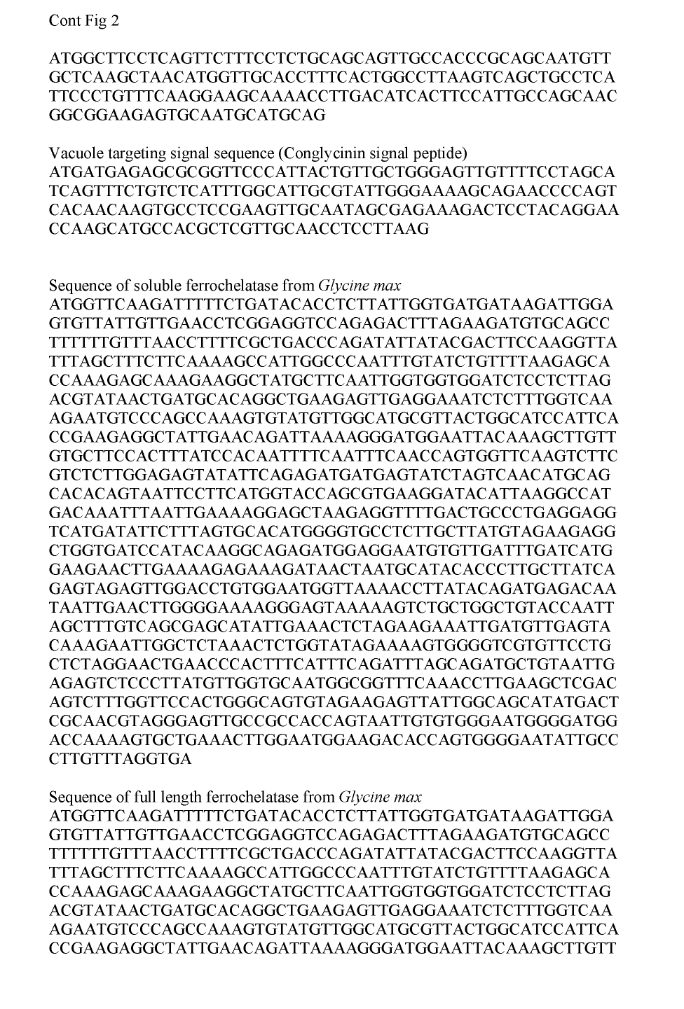

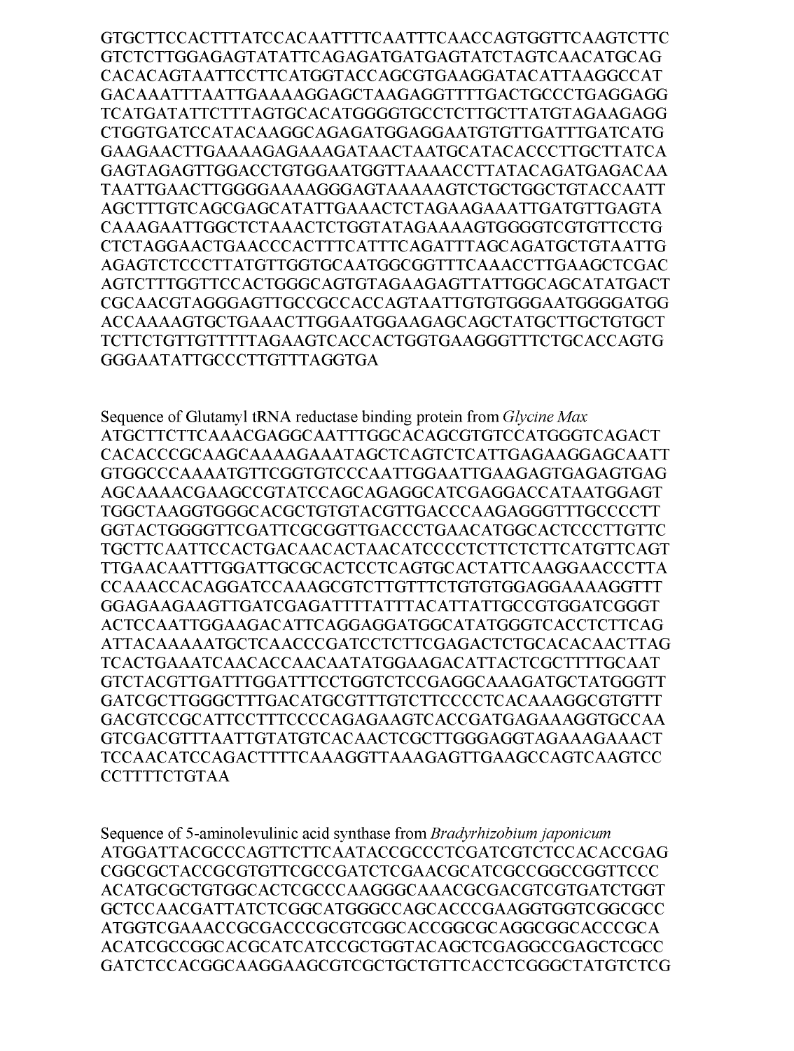

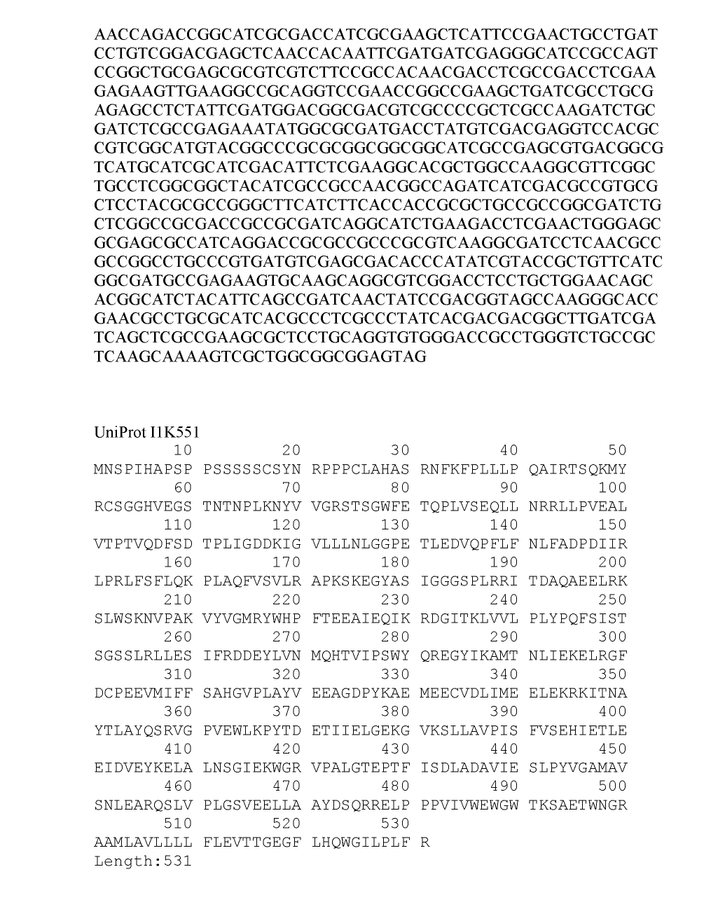

It will be appreciated that the polypeptide that upregulates heme biosynthesis can be a variant (e.g., comprise a mutation such as an amino acid substitution, e.g., a non-conservative or conservative amino acid substitution, an amino acid deletion, an amino acid insertion, or non-native sequence) relative to a wild-type heme biosynthesis polypeptide. For example, a domain such as a transmembrane domain can be removed from a polypeptide that upregulates heme biosynthesis to increase solubility of the polypeptide or a signal peptide can be deleted. For example, a transmembrane domain near the C-terminus of a ferrochelatase (e.g., residues 501-521 of the Glycine max ferrochelatase set forth in SEQ ID NO: 9) can be deleted and/or the signal peptide of the ferrochelatase (residues 1 to 104 of SEQ ID NO: 9) can be deleted. For example, a ferrochelatase polypeptide can include residues 105 to 531 of the amino acid sequence set forth in Uniport I1K551 (see SEQ ID NO: 9, FIG. 2) or can include residues 105 to 500 and 522 to 531 the amino acid sequence set forth in Uniport I1K551 (see SEQ ID NO: 9, FIG. 2). A truncated glutamate tRNA reductase protein can have one or more N-terminal residues (e.g., 5, 10, 15, 20, 25, 30, 35, or 40 residues) removed as described below to, for example, remove feedback inhibition by heme. In some instances, a variant polypeptide can include at most 1, 2, 3, 4, 5, 6, 7, 8, 9, 10, 15, 20, 30, 40, or 50 mutations. In some instances, a variant polypeptide comprises at least 1, 2, 3, 4, 5, 6, 7, 8, 9, 10, 15, 20, 30, 40, or 50 or more mutations. In some instances, at least 1, 2, 3, 4, 5, 6, 7, 8, 9, 10, 20, 30, 40, or 50% of the sequence of a polypeptide of the disclosure can be mutated. In some instances, at most 1, 2, 3, 4, 5, 6, 7, 8, 9, 10, 20, 30, 40, or 50% of the sequence of a polypeptide can be mutated.

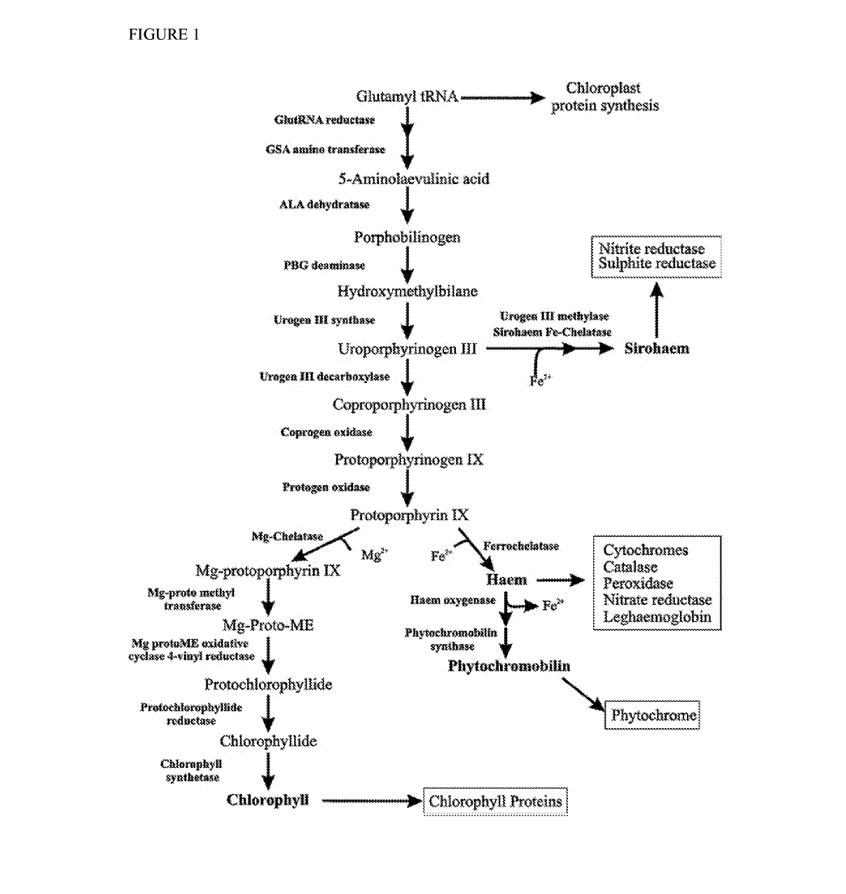

Ferrochelatase is the enzyme at the branchpoint of the biosynthetic pathway that pulls the biosynthetic flux towards heme rather than chlorophyll. See, FIG. 1. Overexpression of a ferrochelatase in combination with a heme protein can increase the heme-loading of the heme polypeptide. In some embodiments, a ferrochelatase lacking a C-terminal transmembrane domain can be used. Non-limiting examples of suitable ferrochelatases include a barley ferrochelatase, a tobacco ferrochelatase, a soy ferrochelatase, a mung bean ferrochelatase (e.g., GenBank Accession No. XP_014509945, C-terminal domain from residues 500-520 can be deleted) or a microbial ferrochelatase (e.g., a Bradyrhizobium ferrochelatase such as Bradyrhyzobium japonicum ferrochelatase (GenBank Accession No. AJA60352.1) or an Aspergillus ferrochelatase such as Aspergillus niger ferrochelatase). As used herein, the term microbial refers to a bacterial, viral, or fungal polypeptide.

In some embodiments, a GluTR binding protein mediates heme biosynthetic flux through a spatially separated system that leads only to heme and avoids some of the feedback loops. See, for example, Czarnecki et al., J. Exp Bot, 63 (4): 1675-1687, 2012. Non-limiting examples of suitable GluTR binding proteins include the soy, Arabidopsis thailiana, barley, Medicago trunculata, adzuki bean, and kidney bean GluTR binding protein. Overexpressing the GluTR binding protein in combination with a heme polypeptide can lead to increased heme-loading of the heme polypeptide.

Glutamyl tRNA reductase converts glutamate molecules that are ligated at their α-carboxyl groups to tRNA_{Glu} into glutamate 1-semialdehyde, an intermediate in the synthesis of 5-aminolevulinate, chlorophyll and heme. GluTR activity is inhibited by binding of heme to its N-terminus (see, e.g., Vothknecht et al., Phytochemistry 47: 513-519, 1998). Removal of the first 30 amino acids of a GluTR (e.g., the soy or barley GluTR) removes the feedback inhibition by heme. Therefore, overexpressing a truncated GluTR protein (e.g., a truncated GluTR protein from Glycine max that is 453 amino acids in length, encoded by SEQ ID NO:2) in combination with a heme polypeptide can lead to increased heme-loading of the heme polypeptide. It will be appreciated that for different glutamyl tRNA reductase polypeptides, the optimal truncation may be greater or less than 30 amino acids.

5-Aminolevulinic acid (ALA), a non-protein amino acid, is the first committed intermediate in the common tetrapyrrole pathway for synthesis of heme, chlorophyll, and cytochrome. In nature, there are two known alternate routes by which this committed intermediate is generated. One route is the C4 pathway (Shemin pathway), which involves the condensation of succinyl-CoA and glycine to ALA by ALA synthase (ALAS). The C4 pathway is restricted to mammals, fungi and purple nonsulfur bacteria. The second route is the C5 pathway, which involves three enzymatic reactions resulting in the biosynthesis of ALA from glutamate. The C5 pathway is active in most bacteria, all archaea and plants. See, e.g., Zhang, et al., Sci Rep., 5:8584 (2015).

Expression of bacterial aminolevulinic acid synthase (ALAS) can increase the flux through the tetrapyrrole biosynthesis pathway as plants have no control mechanism for the C4 route of ALA production. The increased flux can be captured by the overexpressed heme polypeptide, leading to increased heme-loading of the heme polypeptide. The bacterial ALAS can be a Rhodobacter ALAS or rhizobia ALAS such as Bradyrhizobium japonicum ALAS (see SEQ ID NO: 8).

The term “heme containing protein” can be used interchangeably with “heme containing polypeptide” or “heme protein” or “heme polypeptide” or “heme-loaded heme-containing polypeptide” and includes any polypeptide that can covalently or noncovalently bind a heme moiety. The terms “heme cofactor” and “heme” are used interchangeably and refer to a prosthetic group bound to iron (Fe2+ or Fe3+) in the center of a porphyrin ring. In some embodiments, the heme-containing polypeptide is a globin such as one in Pfam 00042 and can include a globin fold, which comprises a series of seven to nine alpha helices. Globin type proteins can be of any class (e.g., class I, class II, or class III), and in some embodiments, can transport or store oxygen. For example, a heme-containing protein can be a non-symbiotic type of hemoglobin or a leghemoglobin.

A heme-containing polypeptide can be a monomer, i.e., a single polypeptide chain, or can be a dimer, a trimer, tetramer, and/or higher order oligomer. The life-time of the oxygenated Fe²⁺ state of a heme-containing protein can be similar to that of myoglobin or can exceed it by 10%, 20%, 30%, 50%, 100% or more under conditions in which the heme-protein-containing consumable is manufactured, stored, handled or prepared for consumption. The life-time of the unoxygenated Fe²⁺ state of a heme-containing protein can be similar to that of myoglobin or can exceed it by 10%, 20%, 30%, 50%, 100% or more under conditions in which the heme-protein-containing consumable is manufactured, stored, handled or prepared for consumption

Non-limiting examples of heme-containing polypeptides can include leghemoglobin, an androglobin, a cytoglobin, a globin E, a globin X, a globin Y, a hemoglobin, a myoglobin (e.g., bovine myoglobin), an erythrocruorin, a beta hemoglobin, an alpha hemoglobin, a protoglobin, a cyanoglobin, a cytoglobin, a histoglobin, a neuroglobin, a chlorocruorin, a truncated hemoglobin (e.g., HbN or HbO), a truncated 2/2 globin, a hemoglobin 3 (e.g., Glb3), a cytochrome, or a peroxidase.

Heme-containing proteins that can produced in the plants, plant cells, and seeds described herein can be from mammals (e.g., farms animals such as cows, goats, sheep, pigs, ox, or rabbits), birds, plants, algae, fungi (e.g., yeast or filamentous fungi), ciliates, or bacteria. For example, a heme-containing protein can be from a mammal such as a farm animal (e.g., a cow, goat, sheep, pig, fish, ox, or rabbit) or a bird such as a turkey or chicken. Heme-containing proteins can be from a plant such as Nicotiana tabacum or Nicotiana sylvestris (tobacco); Zea mays (corn), Arabidopsis thaliana, a legume such as Glycine max (soybean), Cicer arietinum (garbanzo or chick pea), Pisum sativum (pea) varieties such as garden peas or sugar snap peas, Phaseolus vulgaris varieties of common beans such as green beans, black beans, navy beans, northern beans, or pinto beans, Vigna unguiculata varieties (cow peas), Vigna radiata (mung beans), Lupinus albus (lupin), or Medicago sativa (alfalfa); Brassica napus (canola); Triticum sps. (wheat, including wheat berries, and spelt); Gossypium hirsutum (cotton); Oryza sativa (rice); Zizania sps. (wild rice); Helianthus annuus (sunflower); Beta vulgaris (sugarbeet); Pennisetum glaucum (pearl millet); Chenopodium sp. (quinoa); Sesamum sp. (sesame); Linum usitatissimum (flax); or Hordeum vulgare (barley). Heme-containing proteins can be isolated from fungi such as Saccharomyces cerevisiae, Pichia pastoris, Magnaporthe oryzae, Fusarium graminearum, Aspergillus oryzae, Trichoderma reesei, Myceliopthera thermophile, Kluyveramyces lactis, or Fusarium oxysporum. Heme-containing proteins can be isolated from bacteria such as Escherichia coli, Bacillus subtilis, Bacillus licheniformis, Bacillus megaterium, Synechocistis sp., Aquifex aeolicus, Methylacidiphilum infernorum, or thermophilic bacteria such as Thermophilus spp. The sequences and structure of numerous heme-containing proteins are known. See for example, Reedy, et al., Nucleic Acids Research, 2008, Vol. 36, Database issue D307-D313 and the Heme Protein Database available on the world wide web at http://hemeprotein.info/heme.php. In some embodiments, a leghemoglobin can be a soy, pea, or cowpea leghemoglobin.

It will be appreciated that a heme-containing polypeptide can be a variant (e.g., comprise a mutation such as an amino acid substitution, e.g., a non-conservative or conservative amino acid substitution, an amino acid deletion, an amino acid insertion, or non-native sequence) relative to a wild-type heme-containing polypeptide.

### Recombinant Nucleic Acid Constructs

As described herein, the transgenic plants, transgenic plant cells, or transgenic seeds contain at least one recombinant nucleic acid that includes a) a promoter operably linked to a nucleic acid encoding a heme-containing polypeptide and b) a promoter operably linked to a nucleic acid that encodes a polypeptide that specifically upregulates heme biosynthesis. In some embodiments, the promoter operably linked to a nucleic acid encoding the heme-containing polypeptide and the promoter operably linked to a nucleic acid encoding a polypeptide that specifically upregulates heme biosynthesis are on separate nucleic acid constructs.

The recombinant nucleic acid is exogenous to the plant, plant cell, or seed. As used herein, the term “exogenous” with respect to a nucleic acid indicates that the nucleic acid is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. A heterologous polypeptide as used herein refers to a polypeptide that is not a naturally occurring polypeptide in a plant cell, e.g., a transgenic soybean plant transformed with and expressing the coding sequence for a leghemoglobin from an alfalfa plant. In the plants, cells, and seeds described herein, the heme-containing polypeptide being expressed in the plant can be heterologous to the plant. In the plants, cells, and seeds described herein, the polypeptide that upregulates heme biosynthesis that is being expressed in the plant can be heterologous to the plant.

An exogenous nucleic acid also can be a sequence that is native to a plant (i.e., it is endogenous to the plant) and that has been reintroduced into cells of that plant such as a nucleic acid encoding a soybean ferrochelatase being re-introduced into a soybean plant. In the plants, cells, and seeds described herein, the heme-containing polypeptide being expressed in the plant can be endogenous to the plant. In the plants, cells, and seeds described herein, the polypeptide that upregulates heme biosynthesis that is being expressed in the plant can be endogenous to the plant. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

“Isolated nucleic acid” as used herein includes a naturally-occurring nucleic acid, provided one or both of the sequences immediately flanking that nucleic acid in its naturally-occurring genome is removed or absent. Thus, an isolated nucleic acid includes, without limitation, a nucleic acid that exists as a purified molecule or a nucleic acid molecule that is incorporated into a vector or a virus. A nucleic acid existing among hundreds to millions of other nucleic acids within, for example, cDNA libraries, genomic libraries, or gel slices containing a genomic DNA restriction digest, is not to be considered an isolated nucleic acid.

“Nucleic acid” and “polynucleotide” are used interchangeably herein, and refer to both RNA and DNA, including cDNA, genomic DNA, synthetic DNA, and DNA or RNA containing nucleic acid analogs. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

In the recombinant nucleic acid constructs described herein, the promoters can be the same or different. In some embodiments, the recombinant nucleic acid construct can be configured such that the nucleic acid encoding the heme-containing polypeptide and the nucleic acid encoding the polypeptide that upregulates heme biosynthesis are contiguous and a single promoter is used to drive transcription of both nucleic acid sequences. The term “promoter” means a DNA sequence recognized by enzymes/proteins required to initiate the transcription of a specific nucleic acid sequence. A promoter typically refers, e.g., to a sequence of nucleic acid to which an RNA polymerase and/or any associated factors binds and at which transcription is initiated. In some embodiments, the recombinant nucleic includes at least one promoter that is positioned 5′ to the nucleic acid sequence encoding a heme polypeptide or a polypeptide that upregulates heme biosynthesis. As used herein, “operably linked” refers to a segment of DNA being linked to another segment of DNA when placed into a functional relationship with the other segment.

In some embodiments, the promoter can be a seed-specific promoter. For example, a seed specific promoter can be the soy beta-conglycinin gene (see, for example, Chen, et al., Dev Genet., 10 (2):112-22 (1989)), a G1-Glycinin seed specific promoter (Ding, et al., Biotechnol Lett., 28 (12):869-75 (2006)), a KTI promoter (see, for example, Perez-Grau and Goldberg, Plant Cell., 1 (11): 1095-1109 (1989)), or an oleosin promoter such as P24 (see, for example, Keddie, et al., Plant Mol Biol., 24 (2):327-40 (1994)). Other non-limiting examples include promoters from the following seed-genes: zygote and embryo LEC1; suspensor G564; maize MAC1 (see, Sheridan, Genetics 142:1009-1020 (1996)); maize Cat3, (see, GenBank No. L05934, Abler, Plant Mol. Biol. 22:10131-1038, (1993)); Arabidopsis viviparous-1, (see, Genbank No. U93215); Arabidopsis atmycl, (see, Urao, Plant Mol. Biol. 32:571-57 (1996), Conceicao, Plant 5:493-505 (1994)); Brassica napus napin gene family, including napA, (see, GenBank No. J02798, Josefsson BL 26:12196-1301 (1987), and Sjodahl, Planta 197:264-271 (1995)).

In some embodiments, the promoter can be a constitutive promoter such as the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, or a ubiquitin promoter such as the maize ubiquitin-1 promoter.

An inducible promoter can include, for example, a core or basal promoter sequence and one or more elements such as transcriptional activator binding sites or other regulatory element to allow control of transcription. A core promoter refers to the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

For example, an inducible promoter can be a modified cauliflower mosaic virus (CaMV) 35S promoter that is responsive to tetracycline. See, Gatz, et al., Plant J., 2, 397-404 (1992), and Weinmann, et al., Plant J., 5, 559-569 (1994). For example, an inducible promoter can be dexamethasone-inducible, or dexamethasone-inducible and tetracycline-inactivatable. See, Aoyama and Chua, Plant J., 11, 605-612 (1997), Craft, et al., Plant J., 41, 899-918 (2005); Samalova, et al., Plant J., 41, 919-935 (2005); Böhner, et al., Plant J., 19, 87-95 (1999); and Böhner, S. and Gatz, Mol. Gen. Genet. 264, 860-870 (2001). For example, an inducible promoter can be responsive to copper. See, Mett, et al., Proc. Natl Acad. Sci. USA, 90, 4567-4571 (1993). For example, an inducible promoter can be responsive to an insecticide (e.g., tebufenozide or methoxyfenozide). See, Koo, et al., Plant J., 37, 439-448 (2004); Martinez, et al., Plant J., 5, 559-569 (1999); and Padidam, et al., Transgenic Res., 12, 101-109 (2003). For example, an inducible promoter can be estrogen responsive (e.g., 17 beta estradiol). See, Bruce, et al., (2000) Plant Cell, 12, 65-80 (2000); and Zuo, et al., Plant J., 24, 265-273 (2000).

For example, an inducible promoter can be responsive to salicylic acid, ethylene, or jasmonic acid. See, for example, Liu, et al., Plant Biotech. J., 11, 43-52 (2013) and Liu, et al., BMC Biotechnol., 11, 108 (2011). Salicylic acid (SA) interacts with either the Arabidopsis PR1 promoter or SA-responsive elements (SARE), which drives the expression of the nucleic acid of interest. Ethylene (ET) interacts with ethylene responsive element (ERE), which drives the expression of the nucleic acid of interest. Methyl jasmonate interacts with jasmonic acid responsive element (JAR) which drives the expression of the nucleic acid of interest. In some embodiments, the inducer (e.g., ethylene gas) can be added to the malting chamber to induce expression of the nucleic acid.

For example, an inducible promoter can be ethanol responsive (e.g., ethanol or acetaldehyde). See, Caddick, et al., Nat. Biotechnol., 16, 177-180 (1998); Roslan, et al., Plant J., 28, 225-235 (2001); and Salter, et al., Plant J., 16, 127-132 (1998). In the presence of ethanol or acetaldehyde, the Aspergillus nidulans ALCR transcription factor (alcR) drives expression from the palcA promoter by binding to upstream sequences (alcA) from the A. nidulans alcA locus. The palcA promoter is positioned upstream of a target DNA for expression.

In some embodiments, ethanol-inducible expression can be based on inducible release of viral RNA replicons from stably integrated DNA proreplicons. See, Werner, et al., Proc Natl Acad Sci USA, 108 (34): 14061-14066 (2011).

In some embodiments, the promoter can be a germination specific promoter. Such a promoter results in expression of the target product during germination and/or early seedling growth in one or more of the radical, hypocotyl, cotyledons, epicotyl, root tip, shoot tip, meristematic cells, seed coat, endosperm, true leaves, internodal tissue, and nodal tissue. See, for example, promoters from genes encoding the glyoxysomal enzymes isocitrate lyase (ICL) and malate synthase (MS) from several plant species (Zhang et al., Plant Physiol. 104: 857-864, 1994); Reynolds and Smith, Plant Mol. Biol. 27: 487-497, 1995); Comai et al., Plant Physiol. 98: 53-61, 1992). Promoters also can be from other genes whose mRNAs appear to accumulate specifically during the germination process, for example class I β-1,3-glucanase B from tobacco (Vogeli-Lange et al., Plant J., 5: 273-278, 1994); canola cDNAs CA25, CA8, AX92 (Harada et al., Mol. Gen. Genet., 212: 466-473, 1988); Dietrich et al., J. Plant Nutr., 8: 1061-1073, 1992), lipid transfer protein (Sossountzove et al, Plant Cell, 3: 923-933, 1991); or rice serine carboxypeptidases (Washio et al., Plant Phys., 105: 1275-1280, 1994); and repetitive proline rich cell wall protein genes (Dana et al., Plant Mol. Biol. 14: 285-286, 1990). See U.S. Patent Publication No. 20160024512. The α-amylase promoter also can be used a germination specific promoter. See, Eskelin, et al., Plant Biotechnology Journal, 7: 657-672 (2009).

In some embodiments, the nucleic acid construct further includes a targeting sequence that can be used to direct the heme polypeptide and/or heme biosynthesis polypeptide to one of several different intracellular compartments, including, for example, the endoplasmic reticulum (ER), mitochondria, plastids (such as chloroplasts) such as the RuBisCo plastid targeting sequence, the vacuole, the Golgi apparatus, protein storage vesicles (PSV) and, in general, membranes, to structures such as the roots, or cells in, for example, the hypocotyl. For example, the heme polypeptide and/or heme biosynthesis polypeptide can be directed to the same intracellular location or to different intracellular locations. Some signal peptide sequences are conserved, such as the Asn-Pro-Ile-Arg (SEQ ID NO: 10) amino acid motif found in the N-terminal propeptide signal that targets proteins to the vacuole (Marty, Plant Cell, 11: 587-599, 1999). Other signal peptides do not have a consensus sequence per se, but are largely composed of hydrophobic amino acids, such as those signal peptides targeting proteins to the ER (Vitale and Denecke, Plant Cell, 11: 615-628, 1999). Still others do not appear to contain either a consensus sequence or an identified common secondary sequence, for instance the chloroplast stromal targeting signal peptides (Keegstra and Cline, Plant Cell, 11: 557-570, 1999). Chloroplast targeting peptides commonly have a high content of hydroxylated amino acid residues (Ser, Thr, and Pro), lack acidic amino acid residues (Asp and Glu), and tend to form α-helical structures in hydrophobic environments (see, e.g., Shen, et al., Scientific Reports, 7, 46231, 2017). In some embodiments, a chloroplast targeting sequence can be a pea, rice, tobacco, Arabidopsis, or soy rubisco small subunit (rbcS) transit peptide (Van den Broeck, et al., Nature, 313, 358-363, 1985). In some embodiments, a portion of the N-terminus of the rbcS protein can be included in the targeting sequence. For example, a portion of the N-terminal unfolded region (e.g., 18, 19, 20, 21, 22, 23, 24, or 25 amino acids) of the rbcS protein can be included in the chloroplast target sequence (see, e.g., Shen, et al., 2017, supra), for a total length of 50-80 amino acids (e.g., 60, 65, 70, 75 amino acids). Furthermore, some targeting peptides are bipartite, directing proteins first to an organelle and then to a membrane within the organelle (e.g. within the thylakoid lumen of the chloroplast; see Keegstra and Cline, 1999, supra). In addition to the diversity in sequence and secondary structure, placement of the signal peptide is also varied. Proteins destined for the vacuole, for example, can have targeting signal peptides found at the N-terminus, at the C-terminus and at a surface location in mature, folded proteins.

In some embodiments, a nucleic acid construct includes a root targeting sequence such as domain A of the CaMV 35S promoter (e.g., containing a tandem repeat of the sequence TGACG separated by 7 base pairs). See, for example, Benfey, et al., The EMBO Journal, 8 (8):2195-2202, 1989.

In some embodiments, a nucleic acid sequence encoding a soy conglycinin vacuole targeting signal peptide, a soy glycinin vacuole targeting signal peptide, or a plant seed storage protein vacuole targeting signal peptide is used as a targeting sequence.

### Producing Transgenic Plant Cells and Plants

Transgenic plant cells and plants comprising at least one recombinant nucleic acid construct described herein can be produced using a variety of techniques. For example, Agrobacterium-mediated transformation, viral vector-mediated transformation, electroporation, or particle gun transformation can be used for introducing nucleic acids into monocotyledonous or dicotyledonous plants. See, for example, U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

The polynucleotides and constructs described herein can be used to transform a number of monocotyledonous and dicotyledonous plants including, for example, Arabidopsis thaliana, Oryza sativa (rice), Glycine max (soybean), a beet, a sugar beet, parsnip, a bean such as an adzuki, a mung, a pea, a peanut, a lentil, or a garbanzo, a leafy vegetable such as an alfalfa, an arugula, a mustard, or a Brassica, or a grass such as a barley, an oat, a wheat, a corn, a rye, triticale, or spelt.

A transformed cell, callus, tissue, or plant can be identified and isolated by selecting or screening the engineered plant material for particular polypeptides or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, quantitative real-time PCR, or reverse transcriptase PCR (RT-PCR) amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. After a polynucleotide is stably incorporated into a transgenic plant, it can be introduced into other plants using, for example, standard breeding techniques.

A population of transgenic plants can be screened and/or selected for those members of the population that produce the target product. For example, a population of progeny of a single transformation event can be screened for those plants having a desired level of expression of the target polypeptide or nucleic acid encoding the target polypeptide.

A plant or plant cell can be transformed by having a construct integrated into its genome, i.e., can be stably transformed. Stably transformed cells typically retain the introduced nucleic acid with each cell division. A plant or plant cell also can be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. As used herein, a transgenic plant also refers to progeny of an initial transgenic plant provided the progeny inherits the transgene. “Progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅, and F₆ refer to subsequent generations of self- or sib-pollinated progeny of an F₁ plant.

Seeds produced by a transgenic plant can be grown and then selfed (or outcrossed and selfed) to obtain seeds homozygous for the nucleic acid construct.

### Methods of Producing Heme-Loaded Heme Polypeptides

The transgenic plants or plant cells described herein can be grown in a manner suitable for the species under consideration, either in a growth chamber, a greenhouse, or in a field, and then the heme-loaded polypeptide can be isolated. In some embodiments, the heme-loading of recombinant heme-containing polypeptides can be increased using the methods and transgenic plants, cells, and seeds described herein. In some embodiments, overall heme-loaded heme protein concentration can be increased in the transgenic plants, cells, and seeds described herein. In some embodiments, increased heme loading caused by the upregulation of heme biosynthesis can increase overall heme-loaded protein accumulation in the transgenic plants, cells, and seeds described herein. In some embodiments, increased heme biosynthesis allows the accumulation of more heme-loaded protein than a corresponding transgenic plant without the upregulation, even when the corresponding plant produces heme-loaded proteins.

For example, using the methods described herein, the heme-loaded heme-containing polypeptide can be present in one or more plant tissues, e.g., seeds, vegetative tissues, reproductive tissues, or root tissues, at increased levels relative to that of corresponding control plants that do not express the polypeptide that upregulates heme biosynthesis. For example, the heme-loading of a recombinant heme-containing polypeptide can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the heme-loading of the heme-containing polypeptide in a corresponding control plant that does not express the polypeptide that upregulates heme biosynthesis. In some embodiments, the heme loaded heme-containing polypeptide can be at least 0.01%, 0.05%, 1%, 2%, 3%, 4%, 5%, 6%, 7%, 8%, 9%, 10% or more of total seed protein in seeds of the transgenic plant. Similarly, in some embodiments, the heme loaded heme-containing polypeptide can be at least 0.01%, 0.05%, 1%, 2%, 3%, 4%, 5%, 6%, 7%, 8%, 9%, 10% or more of total protein from other plant tissues (e.g., roots, leaves, etc.).

In some embodiments in which an inducible promoter is used, the transgenic seeds can be germinated in a contained system in the presence of an inducer and the heme-loaded polypeptide can be isolated from the seedlings. As used herein, a “contained system” refers to a system for germinating transgenic seeds in bulk in a controlled environment (e.g., controlled temperature and humidity) such as a malting or hydroponic system. Germinating seeds in a greenhouse or under field conditions is not considered to be a contained system. See, for example, the U.S. Provisional Application 62/429,557, filed Dec. 2, 2016; and the PCT application entitled “Producing Recombinant Protein in Contained Systems” and filed on Dec. 1, 2017.

To isolate the heme-loaded heme protein from the transgenic plants, cells, or seeds, the plant material can be processed using any appropriate measure including, for example, grinders, hammer mills, shredders, chippers, screwpress, or high pressure homogenization. The heme-loaded proteins can be separated on the basis of their molecular weight, for example, by size exclusion chromatography, ultrafiltration through membranes, or density centrifugation. In some embodiments, heme-loaded proteins can be separated based on their surface charge, for example, by isoelectric precipitation, anion exchange chromatography, or cation exchange chromatography. Heme-loaded proteins also can be separated on the basis of their solubility, for example, by ammonium sulfate precipitation, isoelectric precipitation, surfactants, detergents or solvent extraction. Heme-loaded proteins also can be separated by their affinity to another molecule, using, for example, hydrophobic interaction chromatography, reactive dyes, or hydroxyapatite.

In some embodiments, heme loaded proteins can be extracted in native form as described in WO 2016/054375. For example, a heme-loaded protein can be extracted from the processed plant material with an aqueous solution containing polyethylene glycol (PEG) (e.g., PEG having a MW of 8000) and, optionally, a flocculant such as an alkylamine epichlorohydrin, to generate an extraction slurry that contains bulk solids and an extract; optionally adjusting the pH of the extraction slurry to a pH of 2 to 10; collecting the extract and adding salt such as magnesium sulfate to form a two-phase mixture, separating the two-phase mixture using, for example, gravity settling or centrifugation (e.g., using a disk stack centrifuge) to generate a PEG phase and a product phase; and collecting and filtering (e.g., microfiltering) the product phase to generate a filtered product phase that contains the protein. The filtered product phase can be concentrated and diafiltered to generate a target product concentrate. A product concentrate can be sterilized, e.g., by UV irradiation, pasteurization, or microfiltration, and dried, e.g., by spray drying or a freeze drying under mild conditions.

The invention will be further described in the following examples, which do not limit the scope of the invention described in the claims.

### EXAMPLES

Examples 1-4—relate to overexpressing a truncated glutamyl tRNA reductase (see FIG. 2) in two different intracellular compartments and leghemoglobin in two different intracellular compartments. Examples 5-10 relate to overexpressing ferrochelatase in two different intracellular compartments and after removal of the transmembrane domain (21 amino acids AAMLAVLLLLFLEVTTGEGFL (SEQ ID NO:11) from residues 501-521, close to the C-terminus, see FIG. 2) to render it soluble and overexpressing leghemoglobin in two different intracellular compartments. The encoded ferrochelatases also lack the intrinsic signal peptide corresponding to residues 1 to 104 of UniProt I1K551 (SEQ ID NO: 9). Examples 11-14—relate to overexpressing a glutamyl tRNA reductase binding protein in two different intracellular compartments and leghemoglobin in two different intracellular compartments. Examples 15-18—relate to overexpressing a 5-aminolevulinic acid synthase from Bradyrhizobium japonicum in two different intracellular compartments and leghemoglobin in two different intracellular compartments.

### Example 1: Transformation of A. thaliana and Identification of Transgenic Plants Overexpressing Soy Leghemoglobin Lbc2 and a Truncated Glutamyl tRNA Reductase Lacking the First 30 N-terminal Amino Acids

The expression cassette for expression of leghemoglobin was synthesized by SGI genomics in two parts. Firstly the nucleic acid encoding a soy leghemoglobin Lbc2 (Glyma20g33290.1) from Glycine max was synthesized behind the 7S (beta conglycinin) promoter from Glycine max. A second expression cassette wherein a nucleic acid (SEQ ID NO: 2) encoding a truncated Glutamyl tRNA reductase from Glycine max was synthesized behind the Glycine max G1-Glycinin promoter. The two cassettes were then assembled head to tail and then cloned into the binary vector, pPTN1138IF, which is a member of the pPZP family of binary vectors (see, for example, Hajdukiewicz, et al., 1994, Plant Mol. Biol., 25:989-94).

The binary vector is introduced into A. thaliana using Agrobacterium-mediated transformation. The pPTN1138 vector carries a bar gene (Thompson et al., 1987, EMBO, 6:2519-23) under the control of the Agrobacterium tumefaciensnopaline synthase promoter (Pnos) and terminated using the 3′ UTR of the nopaline synthase gene. Therefore, selection of transformants is performed using the herbicide, Basta.

The transgenic plants are grown to maturation and the seeds are collected. The plants are monitored for healthy growth and increased accumulation of heme loaded leghemoglobin Lbc2 protein expected in the seed compared to plants without overexpression of truncated glutamyl tRNA reductase.

### Example 2: Transformation of A. thaliana and Identification of Transgenic Plants Overexpressing Soy Leghemoglobin Lbc2 and a Plastid Targeted Truncated Glutamyl rRNA Reductase

The expression cassette for expression of leghemoglobin was synthesized by SGI genomics in two parts. The nucleic acid encoding a soy leghemoglobin Lbc2 (Glyma20g33290.1) from Glycine max was synthesized behind the 7S (beta conglycinin) promoter from Glycine max. A second expression cassette was produced wherein the truncated Glutamyl tRNA reductase from Glycine max was synthesized behind the Glycine max G1-Glycinin promoter and the chloroplast ribulose-1-5-bisphosphate carboxylase/oxygenase small unit plastid targeting sequence. The two cassettes were then assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of truncated glutamyl tRNA reductase.

### Example 3: Transformation of A. thaliana and Identification of Transgenic Plants Overexpressing Soy Leghemoglobin Lbc2 Targeted to the Protein Storage Vesicle and a Truncated Glutamyl rRNA Reductase

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein the truncated Glutamyl tRNA reductase from Glycine max was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of truncated glutamyl tRNA reductase.

### Example 4: Transformation of A. thaliana and Identification of Transgenic Plants Overexpressing Soy Leghemoglobin Lbc2 Targeted to the Protein Storage Vesicle and a Plastid Targeted Truncated Glutamyl rRNA Reductase

The expression cassette for expression of leghemoglobin was synthesized by SGI genomics in two parts. Firstly the nucleic acid encoding a soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein a nucleic acid encoding the truncated Glutamyl tRNA reductase from Glycine max was placed behind the Glycine max G1-Glycinin promoter and the chloroplast ribulose-1-5-bisphosphate carboxylase/oxygenase small unit plastid targeting sequence. The two cassettes were then assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of truncated glutamyl tRNA reductase.

### Example 5: Transformation of A. thaliana and Identification of Transgenic Plants Overexpressing Soy Leghemoglobin Lbc2 and a Soluble Ferrochelatase from Glycine max (UNIPROT I1K551_SOYBN-Ferrochelatase from Glycine max: 21 Amino Acids Removed from Close to C Terminus Terminal to Make the Protein Soluble)

The expression cassette for expression of leghemoglobin was synthesized by SGI genomics in two parts. A nucleic acid encoding a soy leghemoglobin Lbc2 (Glyma20g33290.1) from Glycine max was synthesized behind the 7S (beta conglycinin) promoter from Glycine max. A second expression cassette was synthesized wherein the soluble ferrochelatase from Glycine max was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of soluble ferrochelatase in the cytosol.

### Example 6: Transformation of A. thaliana and Identification of Transgenic Plants Overexpressing Soy Leghemoglobin Lbc2 and a Ferrochelatase from Glycine max (UNIPROT I1K551_SOYBN-Ferrochelatase from Glycine max

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. A second expression cassette was synthesized wherein the ferrochelatase from Glycine max was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of ferrochelatase in the cytosol.

### Example 7: Transformation of A. thaliana and Identification of Transgenic Plants Overexpressing Soy Leghemoglobin Lbc2 and a Plastid Targeted Ferrochelatase from Glycine max (UNIPROT I1K551_SOYBN-Ferrochelatase from Glycine max)

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. A second expression cassette was synthesized wherein the ferrochelatase from Glycine max was placed behind the Glycine max G1-Glycinin promoter and the chloroplast ribulose-1-5-bisphosphate carboxylase/oxygenase small unit plastid targeting sequence. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of ferrochelatase in the chloroplast.

### Example 8: Transformation of A. thaliana and Identification of Transgenic Plants Overexpressing Soy Leghemoglobin Lbc2 Targeted to the Protein Storage Vesicle and a Soluble Ferrochelatase from Glycine max (UNIPROT I1K551_SOYBN-Ferrochelatase from Glycine max: 21 Amino Acids Removed from Close to C Terminus Terminal to Make the Protein Soluble)

The expression cassette for expression of leghemoglobin was synthesized by SGI genomics in two parts. The soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein the soluble ferrochelatase from Glycine max was synthesized behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of soluble ferrochelatase.

### Example 9: Transformation of A. thaliana and Identification of Transgenic Plants Overexpressing Soy Leghemoglobin Lbc2 and a Ferrochelatase from glycine max (UNIPROT I1K551_SOYBN-Ferrochelatase from Glycine max)

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein the nucleic acid encoding a ferrochelatase from Glycine max was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of ferrochelatase in the cytosol.

### Example 10: Transformation of A. thaliana and Identification of Transgenic Plants Overexpressing Soy Leghemoglobin Lbc2 Targeted to the Protein Storage Vesicle and a Plastid Targeted Ferrochelatase (UNIPROT I1K551_SOYBN-Ferrochelatase from Glycine max)

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein a nucleic acid encoding the ferrochelatase from Glycine max was placed behind the Glycine max G1-Glycinin promoter and the chloroplast ribulose-1-5-bisphosphate carboxylase/oxygenase small unit plastid targeting sequence. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of ferrochelatase in the plastid.

### Example 11: Transformation of A. thaliana and Identification of Transgenic Plants Overexpressing Soy Leghemoglobin Lbc2 and a Putative Glutamyl tRNA Reductase Binding Protein (UNIPROT C6TE45_SOYBN—Uncharacterized Protein Glycine max)

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. A second expression cassette was synthesized wherein the nucleic acid encoding a putative glutamyl tRNA reductase binding protein from Glycine max (UNIPROT C6TE45) was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without cytosolic overexpression of putative glutamyl tRNA reductase binding protein.

Example 12: Transformation of Arabidopsis and Identification of Transgenic Plants Overexpressing Soy Leghemoglobin Lbc2 and a Plastid Targeted Putative Glutamyl tRNA Reductase Binding Protein

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. A second expression cassette was synthesized wherein the putative Glutamyl tRNA reductase binding protein from Glycine max was placed behind the Glycine max G1-Glycinin promoter and the chloroplast ribulose-1-5-bisphosphate carboxylase/oxygenase small unit plastid targeting sequence. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of putative Glutamyl tRNA reductase binding protein in the plastid.

Example 13: Transformation of A. thaliana and Identification of Transgenic Plants Overexpressing Soy Leghemoglobin Lbc2 Targeted to the Protein Storage Vesicle and a Putative Glutamyl tRNA Reductase Binding Protein

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein the putative Glutamyl tRNA reductase binding protein from Glycine max was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without cytosolic overexpression of putative Glutamyl tRNA reductase binding protein.

### Example 14: Transformation of A. thaliana and Identification of Transgenic Plants Overexpressing Soy Leghemoglobin Lbc2 Targeted to the Protein Storage Vesicle and a Plastid Targeted Putative Glutamyl tRNA Reductase Binding Protein

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. A nucleic acid encoding the conglycinin signal peptide was added before the Lbc2 coding region to target leghemoglobin expression to the protein storage vacuole. A second expression cassette was synthesized wherein the putative Glutamyl tRNA reductase binding protein from Glycine max was placed behind the Glycine max G1-Glycinin promoter and the chloroplast ribulose-1-5-bisphosphate carboxylase/oxygenase small unit plastid targeting sequence. The two cassettes were assembled head to tail and then cloned into the binary vector, pPTN1138IF.

The binary vector is introduced into rice and Arabidopsis using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of putative glutamyl tRNA reductase binding protein in the plasmid.

### Example 15: Transformation of A. thaliana and Identification of Transgenic Plants Overexpressing Soy leghemoglobin Lbc2 and a 5-Aminolevulinic Acid Synthase from Bradyrhizobium japonicum (UNIPROT A0A03YXD2_BRAJP)

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. A second expression cassette was synthesized wherein the nucleic acid encoding the 5-aminolevulinic acid synthase from Bradyrhizobium japonicum was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without cytosolic overexpression of 5-aminolevulinic acid synthase from Bradyrhizobium japonicum.

### Example 16: Transformation of A. thaliana and Identification of Transgenic Plants Overexpressing Soy Leghemoglobin Lbc2 and a Plastid Targeted 5-Aminolevulinic Acid Synthase from Bradyrhizobium japonicum

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. A second expression cassette was synthesized wherein a nucleic acid encoding the 5-aminolevulinic acid synthase from Bradyrhizobium japonicum was placed behind the Glycine max G1-Glycinin promoter and the chloroplast ribulose-1-5-bisphosphate carboxylase/oxygenase small unit plastid targeting sequence. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of 5-aminolevulinic acid synthase from Bradyrhizobium japonicum in the plastid.

### Example 17: Transformation of A. thaliana and Identification of Transgenic Plants Overexpressing Soy Leghemoglobin Lbc2 Targeted to the Protein Storage Vesicle and a 5-Aminolevulinic Acid Synthase from Bradyrhizobium japonicum

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein a nucleic acid encoding the 5-aminolevulinic acid synthase from Bradyrhizobium japonicum was placed behind the Glycine max G1-Glycinin promoter. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without cytosolic overexpression of 5-aminolevulinic acid synthase from Bradyrhizobium japonicum.

### Example 18: Transformation of A. thaliana and Identification of Transgenic Plants Overexpressing Soy Leghemoglobin Lbc2 Targeted to the Protein Storage Vesicle and a Plastid Targeted 5-Aminolevulinic Acid Synthase from Bradyrhizobium japonicum

The expression cassette for expression of leghemoglobin was synthesized by SGI genomics in two parts. A nucleic acid encoding the soy leghemoglobin Lbc2 (Glyma20g33290.1) from Glycine max was synthesized behind the 7S (beta conglycinin) promoter from Glycine max. The conglycinin signal peptide was added before the Lbc2 coding region to target leghemoglobin expression to the protein storage vacuole. A second expression cassette was synthesized wherein a nucleic acid encoding the 5-aminolevulinic acid synthase from Bradyrhizobium japonicum was placed behind the Glycine max G1-Glycinin promoter and the chloroplast ribulose-1-5-bisphosphate carboxylase/oxygenase small unit plastid targeting sequence. The two cassettes were assembled head to tail and then cloned into the binary vector, pPTN1138IF.

The binary vector is introduced into A. thaliana using Agrobacterium-mediated transformation and transformants are selected using the herbicide Basta.

The transgenic plants are grown to maturation and the seeds are collected. The plants are monitored for healthy growth and increased accumulation of heme loaded leghemoglobin Lbc2 protein expected in the seed compared to plants without overexpression of 5-aminolevulinic acid synthase from Bradyrhizobium japonicum in the plasmid.

### Example 19: Isolation of Leghemoglobin

One kg of seeds are hydrated with potassium phosphate buffer (pH 7.4) in a ratio of 1:4 (w/w) are macerated in a VITA-PREP® 3 blender (Vitamix Corp., Cleveland, Ohio). The extraction is performed for 3 minutes at the highest setting (3 HP motor) maintaining the temperature at less than 30° C. at all times. The pH is adjusted to 7.4 post-grinding, using a 10 M NaOH solution. The homogenate is centrifuged at 3500 g for 5 minutes using a bench top centrifuge (Allegra X15R, SX4750 rotor; Beckman Coulter, Inc., Pasadena, Calif.). The pellet is discarded and the supernatant is collected separately. The soluble protein fraction is then microfiltered using a 0.2 μm modified polyethersulfone (mPES) membrane in a hollow fiber format (KROSFLO® K02E20U-05N; Spectrum Laboratories, Inc., Rancho Dominguez, Calif.). The filtrate from this step (about 3 L) is concentrated using a 5 kDa mPES membrane (MiniKros N02E070-05N; Spectrum Laboratories, Inc.) to about 0.1 L. The partially purified leghemoglobin solution is further purified using Q Fast Flow anion exchange resin (GE Lifesciences). The final leghemoglobin product is concentrated using 3 kDa ultrafiltration and frozen at −20° C.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A transgenic plant comprising at least one recombinant nucleic acid, wherein the recombinant nucleic acid comprises
(i) a first promoter operably linked to a nucleic acid encoding a heme-containing polypeptide and;
(ii) a second promoter operably linked to a nucleic acid encoding a polypeptide that upregulates heme biosynthesis, wherein heme-loading of the heme-containing polypeptide is increased in the transgenic plant relative to that of a corresponding control plant that does not comprise the polypeptide that upregulates heme biosynthesis.

2. The transgenic plant of claim 1, wherein the first and second promoters are the same.

3. (canceled)

4. The transgenic plant of any one of claim 1, wherein the first and second promoters are seed specific promoters.

5. The transgenic plant of claim 4, wherein the seed specific promoter is selected from the group consisting of a soy beta-conglycinin seed specific promoter, a G1-Glycinin seed specific promoter, a Kunitz trypsin inhibitor (KTI) promoter, and an oleosin promoter.

6. The transgenic plant of any one of claim 1, wherein the recombinant nucleic acid further comprises a first targeting sequence operably linked to the nucleic acid encoding the heme-containing polypeptide, and a second targeting sequence operably linked to the nucleic acid encoding the polypeptide that upregulates heme biosynthesis.

7. The transgenic plant of claim 6, wherein the first and second targeting sequences target the polypeptides to the same intracellular location within the transgenic plant.

8. The transgenic plant of any one of claim 6, wherein the first and second targeting sequences are the same.

9. The transgenic plant of claim 6, wherein the first targeting sequence, the second targeting sequence, or both encodes a vacuole targeting signal peptide.

10. The transgenic plant of claim 9, wherein the vacuole targeting signal peptide is a soy conglycinin vacuole targeting signal peptide, a soy glycinin vacuole targeting signal peptide, or a plant seed storage protein vacuole targeting signal peptide.

11. The transgenic plant of claim 6, wherein the first targeting sequence, the second targeting sequence, or both encodes a plastid targeting signal peptide.

12. The transgenic plant of claim 11, wherein the plastid targeting signal peptide is a RuBisCO signal peptide.

13. The transgenic plant claim 6, wherein the first targeting sequence, the second targeting sequence, or both is a soy beta-conglycinin targeting sequence.

14. The transgenic plant of claim 6, wherein the first targeting sequence, the second targeting sequence, or both is a G1-Glycinin targeting sequence.

15. The transgenic plant of claim 1, wherein the polypeptide that upregulates heme biosynthesis is a ferrochelatase, a glutamyl-tRNA reductase (GluTR) binding protein, a truncated glutamate tRNA reductase protein (GTR), an aminolevulinic acid synthase, or a combination of two or more of the polypeptides.

16.-20. (canceled)

21. The transgenic plant of claim 1, wherein the heme-containing polypeptide is a globin polypeptide in Pfam 00042.

22. (canceled)

23. The transgenic plant of claim 21, wherein the globin polypeptide is a leghemoglobin, a non-symbiotic hemoglobin, an androglobin, a cytoglobin, a globin E, a globin X, a globin Y, a hemoglobin, a myoglobin, an erythrocruorin, a beta hemoglobin, an alpha hemoglobin, a protoglobin, a cyanoglobin, a cytoglobin, a histoglobin, a neuroglobin, a chlorocruorin, a truncated hemoglobin, a truncated 2/2 globin, a hemoglobin 3, a cytochrome, or a peroxidase.

24. The transgenic plant of claim 21, wherein the globin polypeptide is expressed in the cytosol of the seeds of the transgenic plant cells.

25. The transgenic plant of claim 21, wherein the globin polypeptide is expressed in the vacuole of the seeds of transgenic plant cells.

26. (canceled)

27. (canceled)

28. The transgenic plant of claim 1, wherein the transgenic plant is selected from the group consisting of: a barley, a wheat, a corn, a rye, an oat, a beet, a sugar beet, a parsnip, a bean, a leafy vegetable, a tuber, and a grass.

29.-51. (canceled)

52. A method of producing a heme-loaded heme polypeptide, the method comprising:
growing a plant cell comprising at least one recombinant nucleic acid,
wherein the recombinant nucleic acid comprises
(i) a first promoter operably linked to a nucleic acid encoding a heme-containing polypeptide and;
(ii) a second promoter operably linked to a nucleic acid encoding a polypeptide that upregulates heme biosynthesis; and wherein heme-loading of the heme-containing polypeptide is increased in the plant cell as compared to a corresponding plant cell that does not comprise the polypeptide that upregulates heme biosynthesis.

53.-75. (canceled)
